(19) United States
(12) Patent Application Publication
(10) Pub. No.: US 2023/0285684 A1
(43) Pub. Date: Sep. 14, 2023

(54) AIR FREE SYRINGE CAP

(71) Applicant: SJJ Solutions B.V., Delft (NL)

(72) Inventors: Jasper SCHOOTSTRA, Delft (NL); Sander SCHOOTSTRA, Delft (NL)

(21) Appl. No.: 17/791,767

(22) PCT Filed: Jan. 8, 2021
(86) PCT No.: PCT/NL2021/050010

§ 371 Date: Jul. 8, 2022

(30) Foreign Application Priority Data
Jan. 9, 2020 (NL) 2024635

(51) Int. Cl. A61M 5/32; A61M 5/315; A61M 5/178

(52) U.S. Cl. CPC A61M 5/3202; A61M 5/31511; A61M 5/1782

(57) ABSTRACT

During filling and capping of a syringe, with for example a fluid medicament, air may be trapped between the syringe, cap and the fluid. This air has to be removed prior to the fluid medicament being injected into the body of a patient. For removing air from the syringe during capping, a cap is provided comprising a protrusion extending from the cap, which protrusion is arranged to be at least partially inserted into an outlet end of the syringe. During this insertion, the protrusion may enter the fluid medicament, and displace the fluid medicament such that excess air is pressed out of the syringe by virtue of the displacement of the fluid medicament. This may prevent traveling of bubbles through the volume of medical liquid in the syringe, which may have to be driven out by tapping or other agitation.

### TECHNICAL FIELD

The invention relates to the field of caps for syringes.

### BACKGROUND

Syringes may be filled with a fluid, such as a liquid medicine. After being filled, a cap is placed on an outlet end of the syringe for sealing the syringe off to prevent the fluid from flowing out.

For filling a syringe, first a plunger of the syringe may be pulled back until the syringe is filled with a desired volume of air. Next, a needle provided on the outlet end of the syringe may be inserted into a vial containing fluid with which the syringe is to be filled. The plunger is then pushed down, pushing the air inside the syringe into the vial. The air prevents a vacuum from forming in the vial which may make it hard to draw out the fluid with the syringe. Pulling back on the plunger again fills the syringe with the fluid from the vial until the syringe has been filled with a desired dose of fluid.

After having filled the syringe with the fluid, some air may be left inside the syringe. Tapping or agitating the syringe may move air bubbles from the fluid to the top of the syringe, and pushing the plunger down while pointing the needle substantially upwards may remove the air from the syringe. Finally, the needle may be removed from the outlet end and a cap may be placed on the outlet end of the syringe for providing a liquid-tight seal for keeping the fluid inside the syringe until the syringe is to be used again.

### SUMMARY

Although syringes may be filled with care, some air bubbles may still be left inside the syringe after capping it, for example due to a cavity inside the cap which is used to seal off the syringe. Furthermore, syringes may be filled by a machine, which machine is not arranged for removing air from the syringe and air bubbles out of the fluid. When a syringe is tapped and/or agitated for moving air bubbles out of the fluid, a silicon oil may be released from the inner wall of the syringe and dissolved into the fluid when a syringe comprising silicon oil is used. Furthermore, a molecular structure of an active substance in the fluid may unwittingly change due to the tapping and/or agitation. Furthermore, agglomerations and/aggregations may occur in the fluid due to the tapping and/or agitation.

It is preferred to provide an improved syringe cap for sealing off an outlet end of a syringe, and/or an improved combination of a syringe and a cap for the syringe. More in particular, it may be preferred to provide a cap that provides for closing a syringe filled with fluid and liquid in particular, the cap being arranged to drive out as much gaseous volume on top of the liquid as possible such that no or only a very limited amount of gaseous substance is present in the closed syringe during storage and transportation, until the moment of use. This prevents traveling of bubbles through the volume of medical liquid in the syringe, which may have to be driven out by tapping or other agitation.

A first aspect provides a syringe cap for sealing off an outlet end of a syringe, comprising a cap body, comprising a cap base, a skirt part comprising a substantially cylindrical wall extending from the cap base and defining, with the cap base, a syringe chamber for receiving at least part of the outlet end of the syringe, wherein the cap body further comprises a protrusion extending from the cap base into the syringe chamber, which protrusion is arranged to be at least partially inserted into the outlet end of the syringe.

A syringe typically comprises a hollow barrel arranged for holding a volume of fluid, and a plunger arranged to be axially moved through the barrel for controlling the volume available for the fluid inside the barrel. The volume of fluid available in the syringe is thus constrained by the barrel, the plunger and the outlet end, wherein any fluid outside the outlet end may be considered to not be held by the syringe any more. Part of this constrained volume may also be occupied by air, for example may fluid only be present up to a certain fluid level, wherein the rest of the volume is occupied by air.

When such a typical syringe is capped with a cap according to the first aspect, the protrusion extending from the cap base into the syringe chamber is at least partially inserted into the outlet end of the syringe. Before being capped, the syringe may be filled with a fluid up to a certain fluid level, which will often be substantially equal to an end face of the outlet end such that as little as possible air is left in the barrel. By inserting the protrusion into the outlet end, any volume of the protrusion inserted into the outlet end replaces volume available for fluid and/or air in the syringe.

If the protrusion is inserted into the outlet end further than the fluid level, a volume of fluid equal to the volume of the protrusion below the fluid level is displaced. This displacement may cause fluid and/or air to be pushed out of the syringe before a seal is established between the cap and the syringe sealing off the outlet end of the syringe. After the seal has been established, substantially no fluid can be pushed out of the syringe anymore.

It is particularly advantageous that air is pushed out of the syringe by the protrusion of the cap by displacing fluid, as air inside the syringe and more in particular air bubbles in the fluid are undesired. For example, when the fluid is a medicine to be injected in a patient's eye, a single bubble of air may have devastating consequences if injected into the eye.

For connecting or engaging with the syringe, an outer wall of the cylindrical wall of the skirt part may be provided with a radially extending connection element for engaging or connecting with the syringe.

The process of capping the syringe with an embodiment of the cap according to the first aspect may comprise an axial movement of the cap relative to the syringe. For keeping the cap in place on the syringe, one or both of a force or multiple keying elements may be used.

A force may for example be a frictional force between two surfaces, a first of which is comprised by the cap and a second of which is comprised by the syringe. One of the surfaces may be comprised by a thread comprised by a first of the cap and the syringe. In such a case, a second of the cap and the syringe may comprise a radially extending engagement element for engaging the thread, wherein the engagement element comprises the second surface. The radially extending connection or engagement element may be a thread itself or shaped as a thread, or may be any other radially extending engagement element arranged to engage with a thread.

An example of using a thread for connecting syringes and caps are so-called Luer locks, which are known in the art as a standardized system for a fluid fitting for a leak-proof connection between two parts.

In another example, a friction fit connection may be used to temporarily fixate the cap on the syringe. For example, the friction fit connection may be a Luer Slip connection. In such example, one or both of the cap and the syringe may comprise a resilient section arranged to elastically deform to provide a liquid-tight seal between the cap and the syringe. The various aspects are thus not limited to Luer locks and relate to syringes and caps with Luer slip couplings as well.

Additionally, or as an option on itself, two or more keying elements may be used which for ensuring the cap remains fixed on the syringe. The keying elements are arranged to be positioned relative to one another such that an axial movement of the cap relative to the syringe is blocked.

An inner wall of the cylindrical wall of the skirt part may be tapered towards the cap body. When the cylindrical wall of the skirt part, and more in particular an inner wall of the cylindrical wall, is tapered towards the cap body, the inner wall comprises a plurality of diameters. One of these diameters may match a diameter of an outer wall of a syringe, and as such an annular seal may be provided with the outer wall of the syringe at the axial location where the diameters correspond to one another. Such an annular seal is preferably one or both of a liquid tight seal and an air tight seat.

The inner wall of the cylindrical wall, or at least a part thereof, mat have a substantially smooth surface. Having substantially no protrusions or indentations in the at least part of the inner wall surface may increase the ease by which the inner wall may form the annular seal with the outer wall of the syringe.

A second aspect provides a syringe/cap combination, comprising a syringe, comprising a barrel defining a hollow chamber for holding a fluid volume, and an outlet end provided at a distal end of the barrel. The syringe/cap combination further comprises a cap according to any of the embodiments of the first aspect, wherein the protrusion of the cap is arranged to be at least partially inserted into the outlet end of the syringe.

In a syringe/cap combination according to the second aspect, when at least part of the cap is inserted into the cap chamber of the barrel, an annular seal may be provided between part of the outer wall of the outlet end and part of the inner wall of the cylindrical wall of the skirt part of the cap, the annular seal sealing off a sealed volume between an outer surface of the outlet end and the cap.

When the annular seal is achieved, a volume of the protrusion inserted in the outlet end may be greater than or equal to the sealed volume. As such, it may be assured that all the air in the barrel is pushed out of the barrel by virtue of inserting the protrusion in the outlet end.

In an embodiment of the syringe/cap combination, the syringe further comprises a shoulder extending substantially outwardly radially from the barrel at an axial distance from the outlet end, and a barrel skirt comprising a cylindrical barrel skirt wall extending from the shoulder away from a proximal end of the barrel, and the barrel skirt defines together with the shoulder a cap chamber for receiving at least part of the cap.

An inner wall of the cylindrical barrel skirt may comprise one or more radially extending protrusions, which protrude into the cap chamber. The radially extending protrusions may be arranged to engage with radially extending protrusion of the cap. As such the one or more radially extending protrusions protruding into the cap chamber may be shaped as one or more threads.

When the radially extending protrusions of both the cap and the syringe are shaped as thread, these threads may be designed to be complementary to one another. As such, they may be rotated into one another to provide an axial translation of the cap relative to the syringe by rotating the cap relative to the syringe to engage the two threads.

The outer wall of the outlet end may be tapered towards the distal end of the syringe. This tapering, optionally combined with a tapering of the cylindrical wall of the skirt part, and more in particular an inner wall of the cylindrical wall, may improve the sealing properties of the cap sealing of the outer end of the syringe.

The tapering of the outer wall of the outlet may be under a first angle, and the tapering of the cylindrical wall of the skirt part of the cap may be under a second angle. The first angle may be larger than, smaller than, or equal to the second angle. Furthermore may one or both of the first angle and the second angle be a non-constant angle.

A syringe/cap combination may be arranged such that the syringe comprises a plunger, comprising at a distal end a plunger end face, the plunger may be arranged to be inserted into the barrel at the proximal end of the barrel, the plunger may be arranged to be axially moved in the barrel between a retracted position and an inserted position. in the retracted position, the plunger end face and at least part of the barrel distal to the plunger end face may define a volume available for fluid in the barrel. In the inserted position, the plunger end face may be substantially aligned with an end face of the outlet end of the syringe such that the volume available for fluid in the barrel may be substantially zero.

As such, a syringe may be provided arranged to be used with the cap according to the first aspect, which as well is arranged such that substantially the full volume of fluid inside the barrel may be pressed out of the barrel by moving the plunger into the inserted position.

A third aspect provides a kit of parts, comprising a syringe/cap combination according to the second aspect, a needle part, comprising a needle hub comprising a hub base and a needle extending through the needle hub, wherein the needle part comprises a hub skirt extending from the needle hub away from the needle, and wherein the hub skirt and the hub base define a hub chamber arranged to receive at least part of the outlet end of the syringe.

As such, the syringe may be filled with a liquid medicine as an example of a fluid and capped using a cap according to the first aspect to substantially remove all the air from the syringe. After for example being transported from a filling location to a use location, the cap may be removed from the syringe and a needle may be connected to the syringe such that the liquid medicine may be injected, with a reduced risk of injecting air with the liquid medicine.

When the needle part has received at least part of the outlet end of the syringe and the syringe is in the inserted position, the plunger end face, end face of the outlet end and the hub base may be substantially aligned. As such, substantially the entire volume of fluid inside the syringe may be pushed out through the needle.

A fourth aspect provides a method for filling and capping a syringe with a fluid medicament, comprising providing a syringe, filling the syringe with a volume of fluid medicament, capping the syringe with a cap, wherein, while capping the syringe, part of the cap is inserted into the volume of fluid medicament such that at least substantially no air is left in the syringe in contact with the fluid medicament. Preferably, in the method according to the fourth aspect a syringe/cap combination according to the second aspect is used. However, for performing the method, also any other suitable syringe/cap combination may be used.

### BRIEF DESCRIPTION OF THE FIGURES

In the figures,

FIGS. 1A and 1B schematically show a combination of a syringe cap and a syringe;

FIG. 2A shows a section view of an embodiment of a cap;

FIG. 2B shows an isometric view of the cap of FIG. 2A;

FIG. 3A shows a section view of another embodiment of a cap;

FIG. 3B shows a section view of a combination of an embodiment of a syringe cap and part of an embodiment of a syringe;

FIG. 4A shows an isometric, sectioned view of part of an embodiment of a syringe;

FIG. 4B shows a section view of a combination of a syringe and a needle part; and

FIG. 4C shows a more detailed view of part of the combination of FIG. 4B.

### DETAILED DESCRIPTION OF THE FIGURES

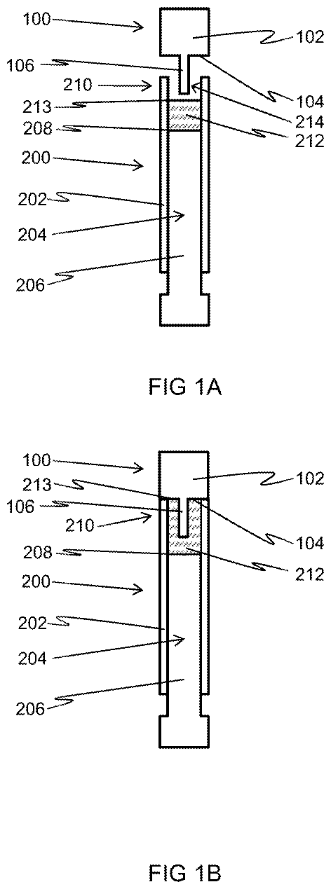

FIGS. 1A and 1B schematically show a combination of a syringe cap 100 and a syringe 200, wherein in FIG. 1A the syringe 200 is not capped with the cap 100, and in FIG. 1B the syringe 200 is capped with the cap 100. Being capped implies that the cap 100 substantially seals off an outlet end 210 of the syringe, such that a substantially liquid-tight and/or air-tight seal may be acquired between the syringe 200 and the cap 100. Such a seal may be used to prevent fluid and/or air from exiting and/or entering the syringe 200, for example during transport of the syringe 200.

The embodiment of the cap 100 as shown in FIGS. 1A and 1B comprises a cap body 102, comprising a cap base 104 and a protrusion 106 extending from the cap base 104 away from the cap body 102. The cap base 104 may thus be a particular surface of the cap body 102. The cap body 102, the protrusion 106, and optionally any other part of an embodiment of the cap 100 may be formed as one solid component.

The embodiment of the syringe 200 as shown in FIGS. 1A and 1B comprises a barrel 202 defining a hollow chamber 204 for holding a fluid volume. Inserted in the barrel 202 and axially translatable in the barrel 202 is a plunger 206 comprising a plunger end face 208. Provided at a distal end of the barrel 202 is an outlet end 210, at which fluid may enter and/or exit the barrel 202. Provided in the hollow chamber 204 in FIG. 1A is a volume of fluid 212 up to a certain fluid level 213 and a volume of air 214.

In FIG. 1B, the syringe 200 is shown capped with the cap 100, i.e. a seal is provided between the cap 100 and the syringe 200 such that the volume of fluid 212 is contained with the syringe. Furthermore, the protrusion 106 of the cap 100 has been inserted into the outlet end 210.

In the transition between FIGS. 1A and 1B, the part of the protrusion 106 which has been inserted below the fluid level 213 caused the fluid level 213 to rise due to the volume of the part of the protrusion 106 occupying the volume previously occupied by the volume of fluid 212. In the example of FIGS. 1A and 1B the fluid level 213 has risen sufficiently that in the state of FIG. 1B, the volume of air 214 has been pressed out of the syringe 200 due to the rising fluid level 213.

Depending on the volume of the protrusion 106, and the expected part of said volume that is to be inserted into the outlet end 210 of the syringe 200, a certain fluid level 213 may be chosen for the filling process of the syringe 200, which may be an automated process performed by a filling machine. The fluid level 213 may be chosen such that it may be ensured that the full volume of air 214 is pressed out of the syringe 200 by virtue of the rising fluid level 213, and the fluid level 213 may even be chosen such that a small volume of fluid is pressed out of the syringe 200 by virtue of the rising fluid level 213 to make even more sure that all air has been pressed out the syringe 200 before the cap 100 seals off the outlet end 210 of the syringe 200.

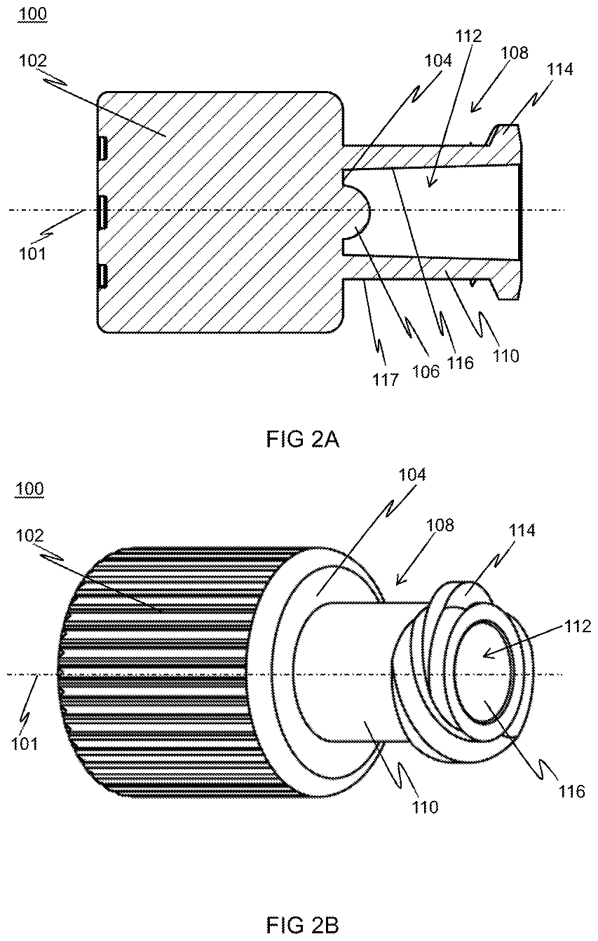

FIG. 2A shows a section view of an embodiment of a cap 100, comprising the cap body 102, cap base 104, and protrusion 106 extending from the cap base 104 away from the cap body 102. The cap 100 as shown in FIG. 2A further comprises, as an option, a skirt part 108 comprising a substantially cylindrical wall 110 extending from the cap base 104 and defining, with the cap base 104, a syringe chamber 112 for receiving at least part of an outlet end 210 of a syringe 200. FIG. 2B shows an isometric view of the cap 100 as shown in FIG. 2A.

The embodiment of the cap 100 as shown in FIG. 2A and FIG. 2B is substantially axisymmetric around a centre line 101. The protrusion 106 is here embodied as being substantially dome-shaped, but may in other embodiments also be differently shaped, for example like a pyramid, rectangle, cone, a frustum of such a shape, any combination thereof or any other protruding shape arranged to be inserted into the outlet end of a syringe.

The shape of the protrusion 106 is preferably tapered away from the cap base 104. Being tapered here may be explained as the shape of the protrusion 106 comprising a larger cross-sectional area perpendicular to the centre line 101 at the cap base 104 than at an end of the protrusion 106 pointing away from the cap base 104. The tapered shape may increase the ease by which the protrusion 106 can be inserted into the outlet end of a syringe, and may assist in self-centring the cap 100 on a syringe 200. The protrusion 106 may further cover the entire surface of the cap base 104, or extend only from part of that surface.

In the embodiments of the cap 100 as shown in FIGS. 2A and 2B, an inner wall 116 of the cylindrical wall 110 of the skirt part 108 is tapered towards the cap body 102. The inner wall 116 furthermore is a substantially smooth surface, meaning that there is no thread or any other significant radially extending protrusion protruding from the inner wall 116 into the syringe chamber 112. With such a smooth surface, it may be possible to receive into the syringe chamber 112 part of a syringe with also a substantially smooth surface.

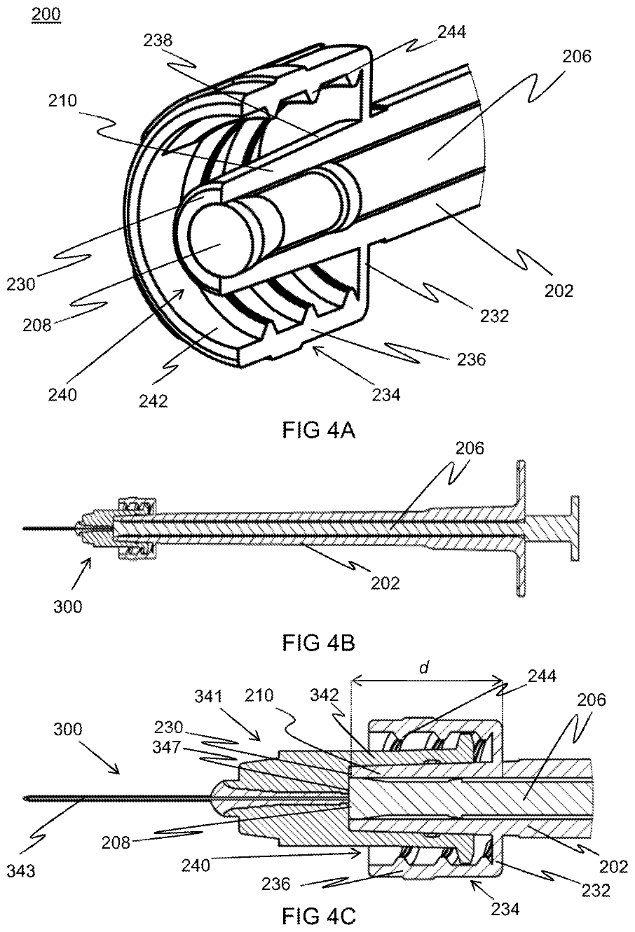

The cap 100 as shown in FIGS. 2A and 2B is provided with a thread 114 as an at least one outwardly radially extending connection element for engaging the syringe 200 at an outer wall 117 of the cylindrical wall 110. The thread 114 may be shaped complementary to a thread comprised by the syringe 200, which may for example be the thread indicated by reference numeral 244 in FIGS. 4A, 4B and 4C.

Alternatively, the outwardly radially extending connection element may be embodied as a radially thickened section extending from the outer wall 117 of the cylindrical wall 110, as one or more flanges extending from the outer wall 117 of the cylindrical wall 110, as any other radially extending protrusion, or as any combination thereof. In embodiments, the outwardly radially extending connection element or at least part thereof may extend from the cap body 102 and/or any other part of the cap 100.

The radially extending connection element allows a connection to be made between the cap and the syringe, provided that the syringe comprises a connection element which is compatible with the connection element comprised by the cap. The connection elements may be arranged to be rotated relative to one another, wherein the rotation results in an axial displacement of the cap relative to the syringe, similar to a threaded screw being screwed into another object. Such an axial displacement may result in the protrusion 106 to be at least partially inserted into the outlet end 210 of the syringe 200.

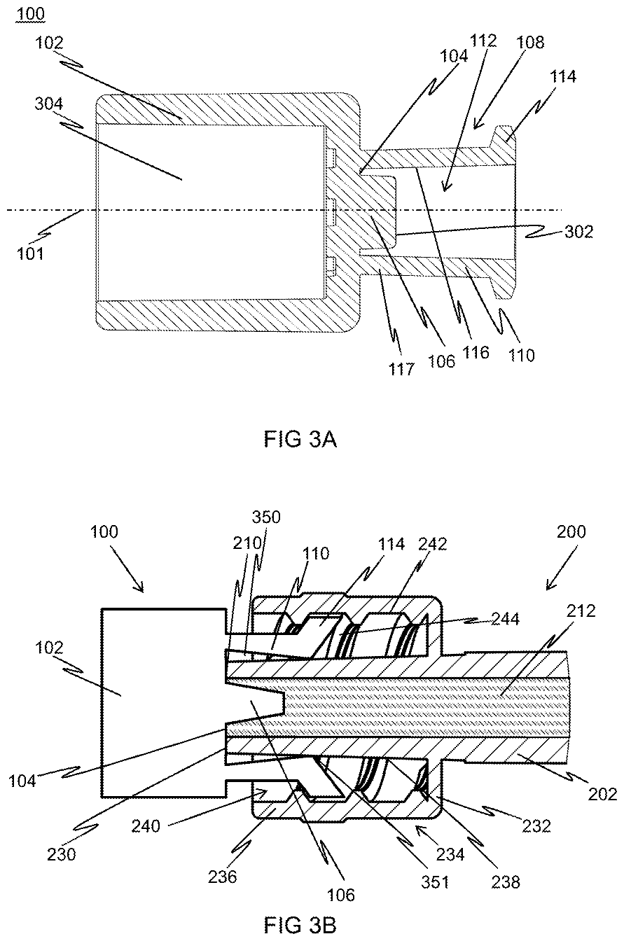

FIG. 3A shows a section view of another embodiment of a cap 100, comprising the cap body 102, cap base 104, and protrusion 106 extending from the cap base 104 away from the cap body 102. The cap 100 as shown in FIG. 2A further comprises a skirt part 108 comprising a substantially cylindrical wall 110 extending from the cap base 104 and defining, with the cap base 104, a syringe chamber 112 for receiving at least part of an outlet end 210 of a syringe 200. The cap 100 as shown in FIG. 3A is substantially axisymmetric around centre line 101. Different options discussed in conjunction with the embodiment of the cap 100 as shown in FIG. 2A may also be applied to the embodiment of the cap 100 as shown in FIG. 3A, and vice versa.

The protrusion 106 as shown in FIG. 3A is, as an option, tapered away from the cap body 102. As such, a diameter of the protrusion 106 at the cap base 104 is larger than a diameter of the protrusion 106 further away from the cap base 104.

As a further option, a distal end-plane 302 of the protrusion 106 is substantially perpendicular to the centre line 101. As an other option, at least part of the distal end-plane 302 of the protrusion 106 is placed at an angle relative to the centre line 101 which is not substantially perpendicular. Different parts of the distal end-plane 302 of the protrusion 106 may be placed at different angles relative to the centre line 101.

The cap body 102 of the cap 100 as shown in FIG. 3A comprises a hollow chamber 304. By virtue of the hollow chamber 304, the material used for manufacturing the cap 100 may be reduced, and the total mass of the cap 100 may be reduced. As an option, the hollow chamber 304 is shaped complementary to the skirt part 108 such that a skirt part 108 of a first cap 100 may be at least partially inserted into the hollow chamber 304 of a second cap 100. This may allow tighter packing of a plurality of caps 100 in a particular volume, for example while shipping a plurality of caps.

FIG. 3B shows a schematic section view of a combination of an embodiment of a syringe cap 100 and part of an embodiment of a syringe 200. The syringe 200 is show capped by the syringe cap 100. Being capped here implies that a seal is provided to the outlet end 210 of the syringe 200 by the cap which prevents or at least substantially prevents.

The syringe 200 comprises a shoulder 232 extending substantially radially from the barrel 202. Extending from the shoulder 232 towards the outlet end 210 is a barrel skirt 234 comprising a cylindrical barrel skirt wall 236. In different embodiments, the barrel skirt 234 may extend beyond the outlet end 210, to the outlet end 210, or the barrel skirt 234 may not extend beyond the outlet end 210. The barrel skirt 234, together with the shoulder 232, defines a cap chamber 240.

In the embodiment of FIG. 3B, an inner wall 242 of the cylindrical barrel skirt wall 236 is provided with a barrel thread 244 as a radially extending protrusion protruding into the cap chamber 240. The cap 100 comprises a cap thread 114 as an at least one outwardly radially extending connection element, and the cap thread 114 is screwed into the barrel thread 244. Whilst screwing the cap thread 114 into the barrel thread 244, the protrusion 106 is inserted further into the barrel 202 and as such the protrusion 106 may displace fluid 212 inside the barrel 202.

In examples, the cap thread 114 can not be screwed further into the barrel thread 244 when the cap base 104 collides with the barrel 202, for example with an outlet end end-face 230. If for example at least one of the cap 100 and the barrel 202 comprise a resilient material which may be elastically deformed by screwing the cap thread 114 further into the barrel thread 244 when the cap base 104 collides with the barrel 202, an annular seal may be obtained which may at least substantially prevent the fluid 212 from exiting the barrel 202.

After the cap 100 is removed from the syringe 200, the volume of the protrusion 106 may be replaced again by air. To remove this air from the syringe 200, the plunger 206 may be pressed down slightly until the air has been removed. The air may be removed prior to or after connecting a needle part to the syringe 200. Although air may thus be present in the syringe 200 after the removal of the cap 100, after connecting the cap 100 to the syringe 200, no air or at least substantially no air is present in the syringe 200.

In embodiments, for example in the embodiment of FIG. 3B, an outer diameter of the protrusion 106 nowhere exceeds an inner diameter of the barrel 202. As such, it may be prevented that the protrusion 106 contacts the barrel 202, for example an inner wall of the barrel 202, and a seal is created prior to the air being displaced out of the barrel 202 by virtue of the rising fluid level caused by the protrusion 106 being inserted into the fluid 212.

As shown in FIG. 3B, with part of the cap 100 inserted in the cap chamber 420, an annular seal 351 may be provided between an outer wall 238 of the outlet end and an inner wall of the cylindrical wall 110. The annular seal 351 substantially seals off of a sealed volume 350 between the cap 100 and the barrel 202. As an option, the volume of the sealed volume 350 is smaller than the volume of the protrusion 106, in particular the volume of the protrusion 106 inserted into the barrel 202.

FIG. 4A shows an isometric, sectioned view of part of an embodiment of a syringe 200, focused on the outlet end 210 of the syringe 200. Provided inside the syringe 200 is the plunger 206 with the plunger end face 208 substantially aligned with a outlet end end-face 230 since the plunger 206 is depicted in an inserted position in FIG. 4A.

The syringe 200 of FIG. 4A further comprises a shoulder 232 extending substantially radially from the barrel 202 at an axial distance (indicated with d in FIG. 4C) from the outlet end 210, more specifically in FIG. 4C from the outlet end end-face 208. Extending from the shoulder 232 away from a proximal end of the barrel is a barrel skirt 234 comprising a cylindrical barrel skirt wall 236. The barrel skirt 234, together with the shoulder 232, defines a cap chamber 240 for receiving at least part of a cap 200.

An outer wall 238 of the outlet end 210, or at least part thereof, is tapered towards the distal end of the syringe 200. The taper angle of the outer wall 238 may be substantially complementary to that of the taper angle of the inner wall 116 of a cap 100. As such, a snug fit may be achieved between the outer wall 238 of the outlet end 210 and the inner wall 116 of the cap 100.

In the embodiment of FIG. 4A, an inner wall 242 of the cylindrical barrel skirt wall 236 is provided with a thread 244 as a radially extending protrusion protruding into the cap chamber 240.

FIG. 4B shows an embodiment of a combination of the syringe 200 of FIG. 4A and a needle part 300, connected to the syringe 200. In FIG. 4C, a more detailed view of part of the combination of the syringe 200 of FIG. 4A and the needle part 300 is depicted.

As depicted in FIG. 4C, the needle part 300 comprises a needle hub 341 comprising a hub base 347 and a needle 343 extending through the needle hub 341. The needle part 300 further comprises a hub skirt 342 extending from the needle hub 341 away from the needle 343. Together, the hub skirt 342 and the hub base 347 define a hub chamber arranged to receive at least part of the outlet end 210 of the syringe 200, as shown in FIG. 4C.

As an option which is shown in FIG. 4C, the plunger 206 is in the inserted position, and the plunger end face 208, the outlet end end-face 230 and the hub base 347 are substantially aligned. With this alignment, no or substantially no volume is available in the hub chamber for air or fluid.

In summary, during filling and capping of a syringe, with for example a fluid medicament, air may be trapped between the syringe, cap and the fluid. This air has to be removed prior to the fluid medicament being injected into the body of a patient. For removing air from the syringe during capping, a cap is provided comprising a protrusion extending from the cap, which protrusion is arranged to be at least partially inserted into an outlet end of the syringe. During this insertion, the protrusion may enter the fluid medicament, and displace the fluid medicament such that excess air is pressed out of the syringe by virtue of the displacement of the fluid medicament. This may prevent traveling of bubbles through the volume of medical liquid in the syringe, which may have to be driven out by tapping or other agitation.

1. A syringe cap for sealing off an outlet end of a syringe, comprising:
a cap body, comprising a cap base;
a skirt part comprising a substantially cylindrical wall extending from the cap base and defining, with the cap base, a syringe chamber for receiving at least part of the outlet end of the syringe;
wherein the cap body further comprises a protrusion extending from the cap base into the syringe chamber, which protrusion is arranged to be at least partially inserted into the outlet end of the syringe.

2. The syringe cap according to claim 1, wherein an outer wall of the cylindrical wall is provided with at least one outwardly radially extending connection element for engaging the syringe.

3. The syringe cap according to claim 2, wherein the radially extending connection element is shaped as a thread.

4. The syringe cap according to claim 1, wherein an inner wall of the cylindrical wall of the skirt part is tapered towards the cap body.

5. The syringe cap according to claim 1, wherein the inner wall of the cylindrical wall has a substantially smooth surface.

6. The syringe cap according to claim 1, wherein the protrusion has at the distal end a substantially continuous convex surface.

7. The syringe cap according to claim 6, wherein the protrusion has at the distal end a substantially continuously convexly curved surface.

8. The syringe cap according to claim 7, wherein the protrusion has at the distal end a substantially hemispherical surface.

9. A syringe/cap combination, comprising:
a syringe, comprising a barrel defining a hollow chamber for holding a fluid volume, and an outlet end provided at a distal end of the barrel; and
a cap according to claim 1;
wherein the protrusion of the cap is arranged to be at least partially inserted into the outlet end of the syringe.

10. The syringe/cap combination according to claim 9, wherein:
the syringe further comprises a shoulder extending substantially outwardly radially from the barrel at an axial distance from the outlet end, and a barrel skirt comprising a substantially cylindrical barrel skirt wall extending from the shoulder away from a proximal end of the barrel; and
the barrel skirt defines together with the shoulder a cap chamber for receiving at least part of the cap.

11. The syringe/cap combination according to claim 10, wherein, when at least part of the cap is inserted into the cap chamber of the barrel, an annular seal is provided between part of the outer wall of the outlet end and part of the inner wall of the cylindrical wall of the skirt part of the cap, the annular seal sealing off a sealed volume between an outer surface of the outlet end and the cap.

12. The syringe/cap combination according to claim 11, wherein when the annular seal is achieved, a volume of the protrusion inserted in the outlet end is greater than or equal to the sealed volume.

13. The syringe/cap combination according to claim 9, wherein an inner wall of the cylindrical barrel skirt wall comprises one or more substantially radially extending protrusions protruding into the cap chamber.

14. The syringe/cap combination according to claim 13, wherein one or more of the one or more radially extending protrusions protruding into the cap chamber are shaped as one or more threads.

15. The syringe/cap combination according to claim 9, wherein an outer wall of the outlet end is tapered towards the distal end of the syringe.

16. The syringe/cap combination according to claim 9, wherein
the syringe comprises a plunger, comprising at a distal end a plunger end face;
the plunger is arranged to be inserted into the barrel at the proximal end of the barrel;
the plunger is arranged to be axially moved in the barrel between a retracted position and an inserted position;
in the retracted position, the plunger end face and at least part of the barrel distal to the plunger end face define a volume available for fluid in the barrel;
in the inserted position, the plunger end face is substantially aligned with an end face of the outlet end of the syringe such that the volume available for fluid in the barrel is substantially zero.

17. A kit of parts, comprising:
a syringe/cap combination according to claim 9;
a needle part, comprising a needle hub comprising a hub base and a needle extending through the needle hub;
wherein the needle part comprises a hub skirt extending from the needle hub away from the needle, and wherein the hub skirt and the hub base define a hub chamber arranged to receive at least part of the outlet end of the syringe.

18. The kit of parts according to claim 17, wherein
the syringe comprises a plunger, comprising at a distal end a plunger end face;
the plunger is arranged to be inserted into the barrel at the proximal end of the barrel;
the plunger is arranged to be axially moved in the barrel between a retracted position and an inserted position;
in the retracted position, the plunger end face and at least part of the barrel distal to the plunger end face define a volume available for fluid in the barrel;
in the inserted position, the plunger end face is substantially aligned with an end face of the outlet end of the syringe such that the volume available for fluid in the barrel is substantially zero, and
wherein, when the needle part has received at least part of the outlet end of the syringe and the plunger is in the inserted position, the plunger end face, the end face of the outlet end and the hub base are substantially aligned.

19. A method for filling and capping a syringe with a fluid medicament, comprising:
providing a syringe;
filling the syringe with a volume of fluid medicament;
capping the syringe with the syringe cap according to claim 1,
wherein, while capping the syringe, part of the cap is inserted into the volume of fluid medicament such that at least substantially no air is left in the syringe in contact with the fluid medicament.
